(19) United States
(12) Patent Application Publication
(10) Pub. No.: US 2024/0390646 A1
(43) Pub. Date: Nov. 28, 2024

(54) MEDICAL APPARATUS

(71) Applicant: CANON KABUSHIKI KAISHA, Tokyo (JP)

(72) Inventors: YUSUKE NIIKAWA, Kanagawa (JP); TOMIO NOGUCHI, Shizuoka (JP)

(21) Appl. No.: 18/791,826

(22) Filed: Aug. 1, 2024

Related U.S. Application Data
(63) Continuation of application No. PCT/JP2023/001640, filed on Jan. 20, 2023.

(30) Foreign Application Priority Data
Feb. 4, 2022 (JP) 2022-016529

(51) Int. Cl. A61M 25/01

(52) U.S. Cl. CPC A61M 25/0113

(57) ABSTRACT

A medical apparatus includes a bending medical device including a bendable body and a base part on which the bendable body is disposed, and a support base including a moving stage configured to support the bending medical device in a detachably attachable manner and a support unit configured to support the moving stage so as to be movable linearly in a predetermined direction, wherein the moving stage supports the bending medical device so as to be movable linearly in the predetermined direction.

### CROSS-REFERENCE TO RELATED APPLICATIONS

This application is a Continuation of International Patent Application No. PCT/JP2023/001640, filed Jan. 20, 2023, which claims the benefit of Japanese Patent Application No. 2022-016529, filed Feb. 4, 2022, both of which are hereby incorporated by reference herein in their entireties.

### BACKGROUND OF THE INVENTION

### Field of the Invention

The present invention relates to a medical apparatus.

### Background Art

There is known a medical apparatus having a bending medical device. In a medical apparatus of Patent Literature 1, when passing a bending medical device through a relatively wide lumen of a patient, an operator detaches the bending medical device from an insertion unit, holds the bending medical device in the operator's hand, and manually inserts the bending medical device into the lumen of the patient. Then, when the bending medical device reaches a delicate site, the operator attaches the bending medical device to the insertion unit and switches to robotic control to continue with the insertion process.

### CITATION LIST

### Patent Literature

Patent Literature 1: United States Patent Application Publication No. 2021/259794

However, in the medical apparatus discussed in Patent Literature 1, in order for an operator to manually insert the bending medical device, an operation by the operator for detaching the bending medical device from the insertion unit is used, and thus there is still a room for improvement in operability.

### SUMMARY OF THE INVENTION

The present invention is directed to improving the operability of a bending medical device.

According to an aspect of the present invention, a medical apparatus includes a bending medical device including a bendable body and a base part on which the bendable body is disposed, and a support base including a moving stage configured to support the bending medical device in a detachably attachable manner and a support unit configured to support the moving stage so as to be movable linearly in a predetermined direction, wherein the moving stage supports the bending medical device so as to be movable linearly in the predetermined direction.

According to the present invention, the operability of a bending medical device can be improved.

Further features of the present invention will become apparent from the following description of exemplary embodiments with reference to the attached drawings.

### BRIEF DESCRIPTION OF THE DRAWINGS

FIG. 1 is an overall view of a medical apparatus according to an exemplary embodiment.

FIG. 2 is a perspective view of a bending medical device and a support base according to the exemplary embodiment.

FIG. 3A is a diagram illustrating an operation of the support base according to the exemplary embodiment.

FIG. 3B is a diagram illustrating the operation of the support base according to the exemplary embodiment.

FIG. 4 is a perspective view of a configuration of the bending medical device according to the exemplary embodiment.

FIG. 5A is a cross-sectional view of a base unit and a moving stage according to the exemplary embodiment.

FIG. 5B is a cross-sectional view of the base unit and the moving stage according to the exemplary embodiment.

FIG. 5C is a cross-sectional view of the base unit and the moving stage according to the exemplary embodiment.

FIG. 6A is a diagram illustrating a configuration of engaging teeth and movable engaging teeth according to the exemplary embodiment.

FIG. 6B is a diagram illustrating the configuration of the engaging teeth and the movable engaging teeth according to the exemplary embodiment.

### DESCRIPTION OF THE EMBODIMENTS

Hereinafter, exemplary embodiments of the present invention will be described with reference to the drawings. The dimensions, materials, shapes, arrangements, and the like of the components described in the exemplary embodiments should be changed as appropriate depending on a configuration of an apparatus to which the present invention is applied, various conditions, and others.

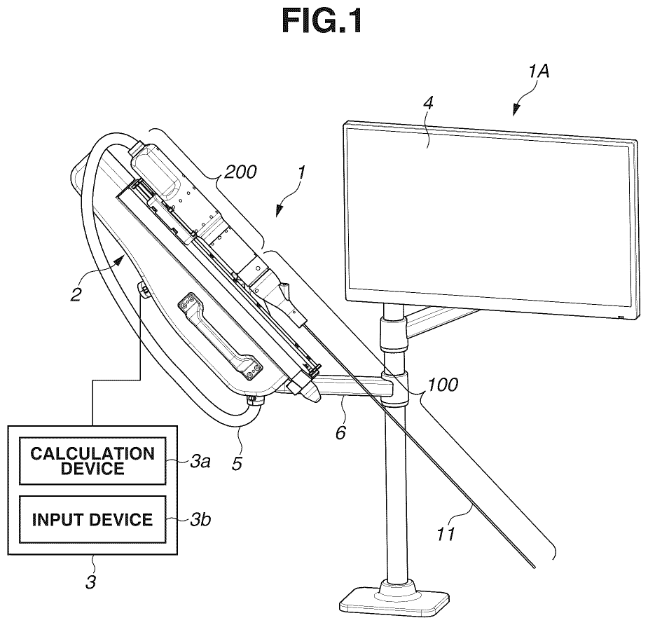

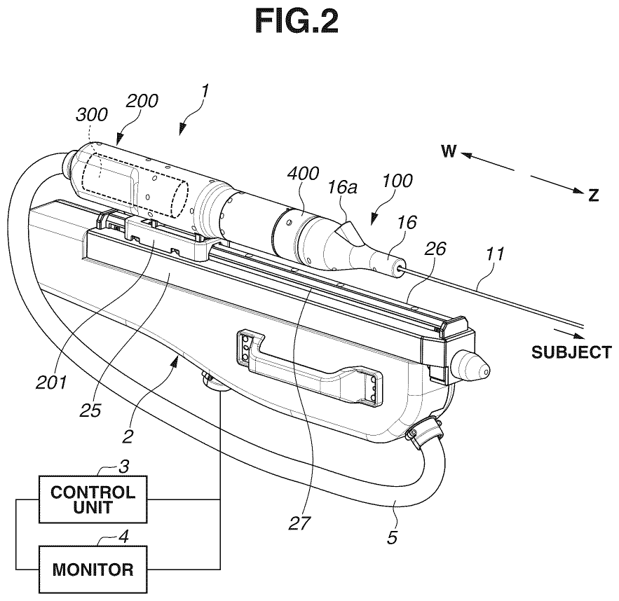

A medical apparatus 1A according to an exemplary embodiment will be described with reference to FIGS. 1 and 2. FIG. 1 is an overall view of the medical apparatus 1A. FIG. 2 is a perspective view of a bending medical device 1 and a support base 2.

The medical apparatus 1A includes the bending medical device 1, the support base 2 that supports the bending medical device 1, and a control unit 3 that controls the bending medical device 1 and the support base 2. The medical apparatus 1A also includes a monitor 4 as a display device, and an arm 6 that supports the monitor 4 and the support base 2.

The bending medical device 1 includes a catheter unit (bendable unit) 100 including a catheter 11 as a bendable body, and a base unit (driving unit or attached unit) 200 that serves as a base portion. The catheter unit 100 is configured to be attachable to and detachable from the base unit 200.

In the present exemplary embodiment, an operator of the medical apparatus 1A and the bending medical device 1 can perform operations, such as observing the inside of a subject, taking various samples from the inside of the subject, and applying treatment to the inside of the subject by inserting the catheter 11 into the inside of the subject. In one exemplary embodiment, the operator can insert the catheter 11 into the inside of a patient as the subject. Specifically, by inserting the catheter 11 into a bronchus through the patient's oral cavity or nasal cavity, the operator can perform operations such as observing, sampling, and excising on lung tissue.

The catheter 11 can be used as a guide (sheath) for guiding a medical tool for performing the above-mentioned operations. Examples of the medical tool include an endoscope, forceps, an ablation device, and the like. Alternatively, the catheter 11 may have the function of the above-mentioned medical tool.

In the present exemplary embodiment, the control unit 3 includes a calculation device 3a and an input device 3b. The input device 3b receives commands and inputs for operating the catheter 11. The calculation device 3a includes a storage for storing programs and various kinds of data for controlling the catheter 11, a random access memory, and a central processing unit for executing the programs. The control unit 3 may also include an output unit that outputs a signal for displaying an image on the monitor 4.

As illustrated in FIG. 2, in the present exemplary embodiment, the bending medical device 1 is electrically connected to the control unit 3 via a cable 5 coupling the base unit 200 and the support base 2 of the bending medical device 1. Alternatively, the bending medical device 1 and the control unit 3 may be directly connected by a cable. The bending medical device 1 and the control unit 3 may be connected wirelessly.

The bending medical device 1 includes a wire driving unit 300 for driving the catheter 11. In the present exemplary embodiment, the bending medical device 1 is a robotic catheter device that drives the catheter 11 by the wire driving unit 300 controlled by the control unit 3.

The control unit 3 controls the wire driving unit 300 to perform an operation of bending the catheter 11. In the present exemplary embodiment, the wire driving unit 300 is built into the base unit 200.

In an extending direction of the catheter 11, an end where a tip of the catheter 11 to be inserted into a subject is located is called a distal end. In the extending direction of the catheter 11, an end opposite to the distal end is called a proximal end.

The catheter unit 100 has a proximal end cover 16 that covers a proximal end side of the catheter 11. The proximal end cover 16 has a tool hole 16a. A medical tool can be inserted into the catheter 11 through the tool hole 16a.

As described above, in the present exemplary embodiment, the catheter 11 functions as a guide device for guiding a medical tool to a desired position inside a subject.

For example, with an endoscope being inserted into the catheter 11, the catheter 11 is inserted up to a target position inside a subject. After the catheter 11 reaches the target position, the endoscope is pulled out of the catheter 11 through the tool hole 16a. Then, the medical tool is inserted through the tool hole 16a to perform operations, such as collecting various specimens from the inside of the subject or applying treatment to the inside of the subject.

As described below, the catheter unit 100 is detachably attached to the base unit 200. After the bending medical device 1 has been used, the operator can detach the catheter unit 100 from the base unit 200, and attach a new catheter unit 100 to the base unit 200 to use the bending medical device 1 again.

The bending medical device 1 includes an operation unit 400. In the present exemplary embodiment, the operation unit 400 is disposed in the catheter unit 100. The operation unit 400 is operated by the operator when the catheter unit 100 is fixed to the base unit 200 or the catheter unit 100 is detached from the base unit 200.

Connecting an endoscope inserted into the catheter 11 and the monitor 4 makes it possible to display an image captured by the endoscope on the monitor 4. In addition, connecting the monitor 4 and the control unit 3 makes it possible to display a state of the bending medical device 1 and information related to control of the bending medical device 1 on the monitor 4. For example, the position of the catheter 11 inside the subject and information related to navigation of the catheter 11 inside the subject can be displayed on the monitor 4. The monitor 4, the control unit 3, and the endoscope may be connected in a wired or wireless manner. Furthermore, the monitor 4 and the control unit 3 may be connected via the support base 2.

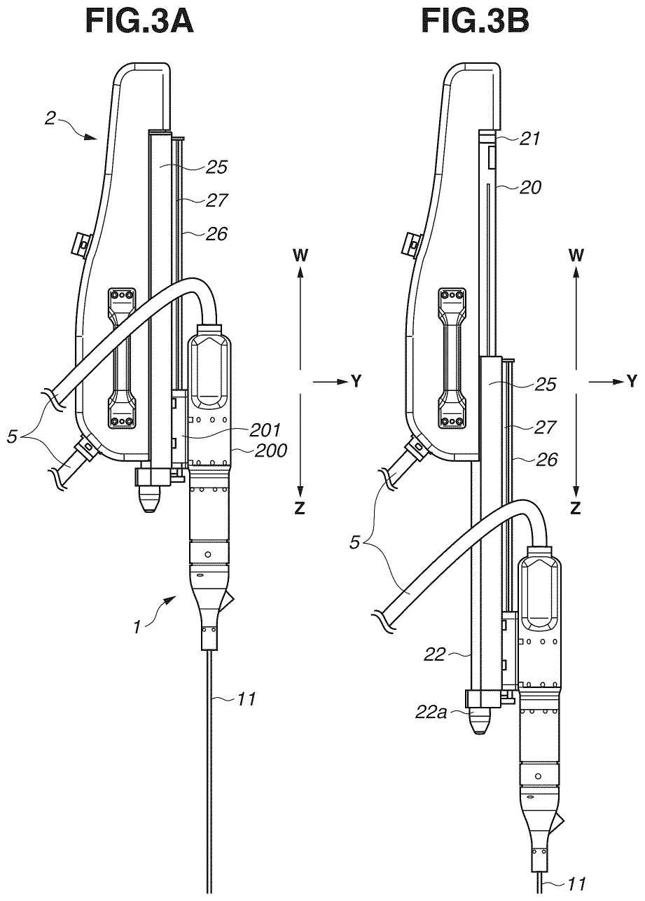

Next, the support base 2 will be described with reference to FIGS. 3A and 3B. FIGS. 3A and 3B are diagrams illustrating operation of the support base 2 according to the present exemplary embodiment. As illustrated in FIGS. 3A and 3B, the support base 2 includes a moving stage 25 having an elongated shape in a Z direction (and a W direction) and a support unit 20 that supports the moving stage 25 to be linearly movable. The support unit 20 includes a motor 21 that is a drive source connected to the control unit 3, and uses a driving force of the motor 21 to perform operations of protruding a cylinder 22 in the Z direction and retracting the cylinder 22 in the W direction opposite to the Z direction. The cylinder 22 is joined to the moving stage 25 at a tip 22a in the Z direction. Therefore, the moving stage 25 can be moved in the Z direction (and the W direction) by the support unit 20.

The bending medical device 1 is detachably attached to the support base 2 via the base unit 200. More specifically, in the bending medical device 1, an attachment portion (connection portion) 201 of the base unit 200 is detachably attached to a guide rail 26 disposed on the moving stage 25 of the support base 2. Even in a state where the attachment portion 201 of the bending medical device 1 is detached from the moving stage 25, connection between the bending medical device 1 and the control unit 3 is maintained so that the bending medical device 1 can be controlled by the control unit 3. In the present exemplary embodiment, even in the state where the attachment portion 201 of the bending medical device 1 is detached from the moving stage 25, the bending medical device 1 and the support base 2 are connected via the cable 5.

The guide rail 26 is disposed over an entire longitudinal area of the moving stage 25 in the Z direction. The operator can attach the bending medical device 1 to the guide rail 26 at any position in the Z direction. For example, the bending medical device 1 may be attached to a tip of the guide rail 26 in the W direction as illustrated in FIG. 2, or the bending medical device 1 may be attached to a tip of the guide rail 26 in the Z direction as illustrated in FIG. 3A.

When the moving stage 25 moves with the bending medical device 1 attached to the moving stage 25, the bending medical device 1 moves. The moving stage 25 then moves in a direction in which the catheter 11 is inserted into the subject (Z direction), and in a direction in which the catheter 11 is pulled out of the subject (W direction). The movement of the moving stage 25 is driven by the motor 21 controlled by the control unit 3.

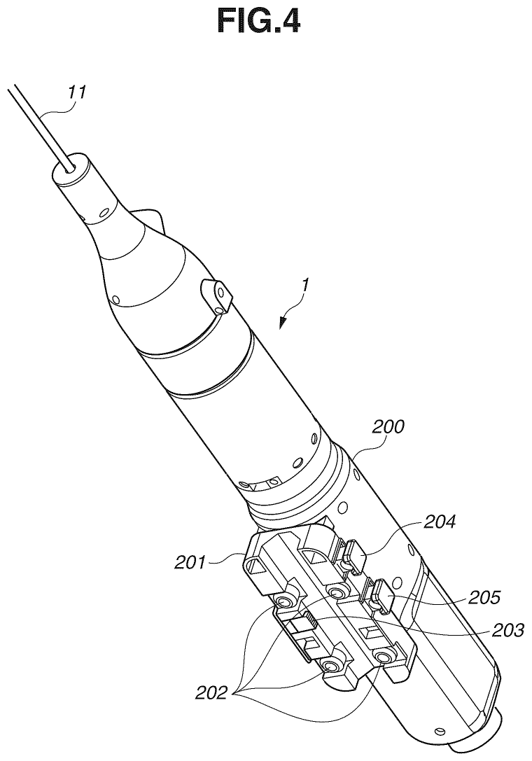

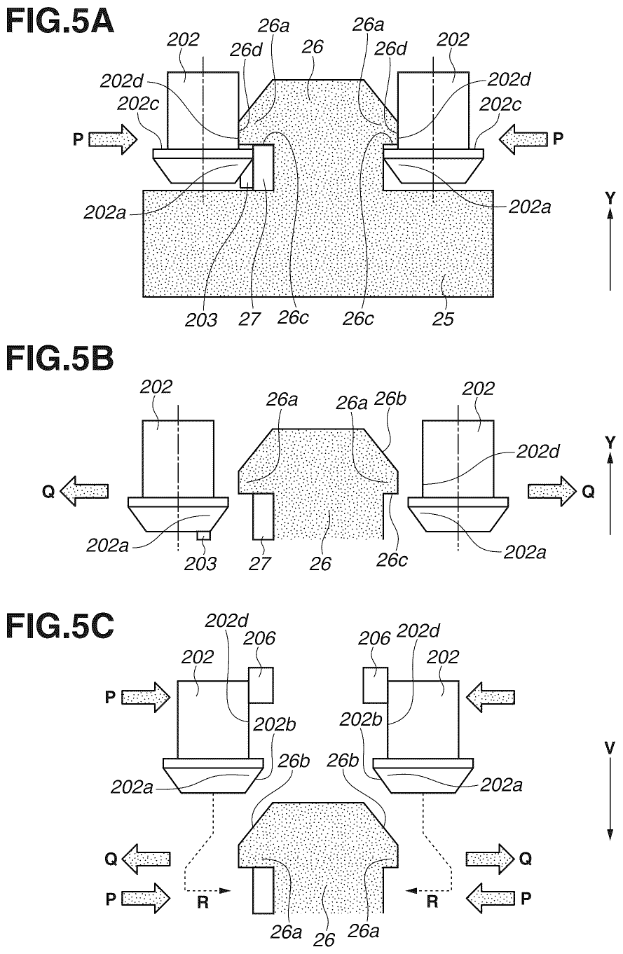

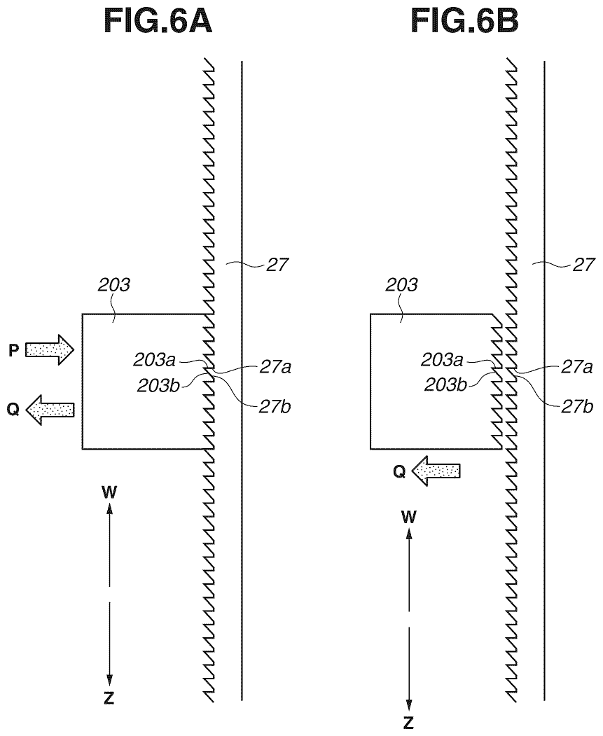

The attachment portion 201 of the base unit 200 is supported in a detachably attachable and movable manner by the guide rail 26 of the moving stage 25. A support configuration of the attachment portion 201 will be described with reference to FIGS. 4, 5A, 5B, 5C, 6A, and 6B. FIG. 4 is a perspective view of the bending medical device 1 according to the present exemplary embodiment. FIGS. 5A, 5B, and 5C are diagrams for describing an attachment/detachment configuration of the bending medical device 1 with respect to the moving stage 25 according to the present exemplary embodiment. FIGS. 6A and 6B are diagrams for describing a movement configuration of the bending medical device 1 with respect to the moving stage 25 according to the present exemplary embodiment.

First, the configuration in which the attachment portion 201 of the base unit 200 is supported in a detachably attachable manner by the guide rail 26 of the moving stage 25 will be described.

FIGS. 5A, 5B, and 5C are cross-sectional views of the attachment portion 201 of the base unit 200 and the guide rail 26 of the moving stage 25 taken along a plane perpendicular to the Z direction. FIG. 5A illustrates a state where the bending medical device 1 is attached to the guide rail 26 of the moving stage 25. The guide rail 26 has protrusions 26a on the left and right sides. As illustrated in FIG. 4, the attachment portion 201 of the base unit 200 includes a plurality of pressing rollers 202 (four in this example). The pressing rollers 202 lock the bending medical device 1 so as not to be detached from the moving stage 25. As illustrated in FIG. 5A, when the bending medical device 1 is attached to the guide rail 26 of the moving stage 25, the pressing rollers 202 are arranged to face each other on both the left and right sides of the guide rail 26. The pressing rollers 202 have protrusions 202a.

The pressing rollers 202 are pressed in P directions by pressing members (springs) not illustrated so as to sandwich the guide rail 26. Due to the pressing, side surfaces 202d of the pressing rollers 202 and side surfaces 26d of the protrusions 26a of the guide rail 26 are in contact with each other. As illustrated in FIG. 5A, when the bending medical device 1 is attached to the guide rail 26 of the moving stage 25, the protrusions 26a of the guide rail 26 and the protrusions 202a of the pressing rollers 202 are engaged with each other. Thus, even if an attempt is made to detach the bending medical device 1 in a Y direction with the protrusions 26a and the protrusions 202a engaged with each other, surfaces 26c of the protrusions 26a of the guide rail 26 abut on surfaces 202c of the protrusions 202a of the pressing rollers 202, so that the bending medical device 1 cannot be detached. In other words, the bending medical device 1 is locked so as not to be detached from the guide rail 26.

As illustrated in FIG. 4, on the attachment portion 201 of the base unit 200, an attachment/detachment unlock button 204, which is an attachment/detachment unlock member, is disposed. The attachment/detachment unlock button 204 is connected to the pressing rollers 202 by a link mechanism (not illustrated). When the attachment/detachment unlock button 204 is pressed, the pressing rollers 202 move in Q directions against pressing forces of the springs in the P directions as illustrated in FIG. 5B. Therefore, the protrusions 26a of the guide rail 26 and the protrusions 202a of the pressing rollers 202 are disengaged, and the bending medical device 1 can be detached in the Y direction from the guide rail 26 (i.e., the support base 2).

An operation of attaching the unattached bending medical device 1 to the support base 2 in a V direction will be described with reference to FIG. 5C. The attachment portion 201 of the base unit 200 includes stoppers 206. When the pressing rollers 202 are pressed in the P directions, the side surfaces 202d abut on the stoppers 206 to determine the positions of the pressing rollers 202 in the P direction.

When the bending medical device 1 is moved in the V direction to be attached to the guide rail 26, inclined surfaces 202b of the protrusions 202a of the pressing rollers 202 come into contact with inclined surfaces 26b of the protrusions 26a of the guide rail 26. When the bending medical device 1 is further moved in the V direction, the pressing rollers 202 spread out in the Q directions along the inclined surfaces 26b of the guide rail 26 against the pressing forces of the springs in the P directions. When the pressing rollers 202 have moved beyond the inclined surfaces 26b of the protrusions 26a of the guide rail 26, the pressing rollers 202 move in the P directions due to the pressing forces of the springs, and reach the state illustrated in FIG. 5A. Accordingly, the bending medical device 1 is attached to the guide rail 26 (i.e., the support base 2). In FIG. 5C, paths taken by the pressing rollers 202 during the attachment thereof are indicated with dashed arrows R.

Next, a configuration in which the attachment portion 201 of the base unit 200 is movably supported in the Z direction (and W direction) by the guide rail 26 of the moving stage 25 will be described. As illustrated in FIG. 2, the guide rail 26 of the moving stage 25 includes engaging teeth 27 over the entire longitudinal area in the Z direction. In addition, as illustrated in FIG. 4, the attachment portion 201 of the base unit 200 includes movable engaging teeth 203. Configurations and operations of the engaging teeth 27 and the movable engaging teeth 203 will be described with reference to FIGS. 6A and 6B that are cross-sectional views taken along a plane perpendicular to the Y direction in FIGS. 5A, 5B, and 5C.

FIG. 6A illustrates a state in which the bending medical device 1 is attached to the support base 2. As illustrated in FIG. 6A, the engaging teeth 27 include a large number of teeth. The engaging teeth 27 are each formed of a surface 27a perpendicular to the Z direction and a slope 27b. The movable engaging teeth 203 are movable in the P (and Q) direction perpendicular to the Z direction, and are disposed at positions facing the engaging teeth 27. The movable engaging teeth 203 are pressed in the P direction by a spring (not illustrated) (spring different from the ones described above). The movable engaging teeth 203 are each formed of a surface 203a perpendicular to the Z direction and a slope 203b.

When the bending medical device 1 is attached to the support base 2, the engaging teeth 27 and the movable engaging teeth 203 are engaged with each other as illustrated in FIG. 6A. More specifically, the engaging teeth 27 and the movable engaging teeth 203 are in contact with each other by surfaces 27a and surfaces 203a engaged with each other and by slopes 27b and slopes 203b engaged with each other.

When the engaging teeth 27 and the movable engaging teeth 203 are engaged with each other, even if an attempt is made to move the bending medical device 1 in the Z direction relative to the moving stage 25, the engaging teeth 27 and the movable engaging teeth 203 restrict a movement by the surfaces 27a and the surfaces 203a, which are perpendicular to the Z direction, coming into contact with each other. Therefore, the bending medical device 1 cannot be moved in the Z direction relative to the moving stage 25.

In order to move the bending medical device 1 in the W direction, a certain amount of force or more is applied to the bending medical device 1 in the W direction. At this time, since the engaging teeth 27 and the movable engaging teeth 203 are in contact with each other by the slopes 27b and the slopes 203b, the movable engaging teeth 203 run on the slopes 27b of the engaging teeth 27 and move in the Q direction against the pressing force of the spring (not illustrated) in the P direction. At this time, since there is nothing to restrict the movement of the movable engaging teeth 203, the bending medical device 1 can move in the W direction. In other words, the engaging teeth 27 and the movable engaging teeth 203 constitute a ratchet, so that the bending medical device 1 is locked so as not to move in the Z direction but is movable in the W direction.

As illustrated in FIG. 4, on the attachment portion 201 of the base unit 200, a movement unlock button 205, which is a movement unlock member, is disposed. The movement unlock button 205 is connected to the movable engaging teeth 203 by a link mechanism (not illustrated). Accordingly, when the movement unlock button 205 is pressed, the movable engaging teeth 203 move in the Q direction against the pressing force of the spring in the P direction, as illustrated in FIG. 6B. Therefore, the engaging teeth 27 and the movable engaging teeth 203 are disengaged from each other, and the bending medical device 1 can be freely moved in the Z direction and the W direction relative to the guide rail 26 of the moving stage 25.

The movable engaging teeth 203 can be disengaged from the engaging teeth 27 due to movement in the Q direction not only by pressing the movement unlock button 205 but also by pressing the attachment/detachment unlock button 204. In other words, the attachment/detachment unlock button 204 is connected to not only the pressing rollers 202 but also the movable engaging teeth 203 by a link mechanism (not illustrated). Thus, when the attachment/detachment unlock button 204 is pressed in the state illustrated in FIG. 5A, the pressing rollers 202 and the movable engaging teeth 203 move in the Q directions as illustrated in FIG. 5B. This disengages the protrusions 26a of the guide rail 26 and the protrusions 202a of the pressing rollers 202 from each other and disengages the engaging teeth 27 and the movable engaging teeth 203 from each other, so that the bending medical device 1 can be detached from the support base 2 in the Y direction.

On the other hand, the movement unlock button 205 is not connected to the pressing rollers 202 by the link mechanism, and even if the movement unlock button 205 is pressed, the pressing rollers 202 do not move in the Q directions, so that the bending medical device 1 does not unexpectedly come off the guide rail 26. Therefore, it is possible to safely move the bending medical device 1 in the Z direction and the W direction.

As described above, the operator inserts the catheter 11 into the inside of a patient as the subject. Specifically, by inserting the catheter 11 into the bronchus through the patient's oral cavity or nasal cavity, the operator can perform operations such as observing, sampling, and excising on lung tissue. There are two methods available for inserting the catheter 11 into the inside of the patient.

One is a method by which the operator presses the movement unlock button 205 disposed on the attachment portion 201 of the base unit 200 to move the bending medical device 1 on the guide rail 26 illustrated in FIGS. 3A and 3B. Hereinafter, this method will be referred to as manual insertion. The other is a method by which the support unit 20 of the support base 2 moves the moving stage 25 to which the bending medical device 1 is attached, linearly in the Z direction. The movement by the support unit 20 is driven by the motor 21 connected to the control unit 3 under an instruction from the operator. Hereinafter, this method will be referred to as robotic insertion.

Switching between the two insertion methods, the manual insertion and the robotic insertion, enables flexible adaptation of insertion of the catheter 11 depending on the required situation. For example, when the catheter 11 is initially inserted through a large or wide portion of the patient's anatomy, the manual insertion can facilitate rapid advancement of the catheter 11, thereby saving time. Once the catheter 11 reaches a more tortuous portion of the patient's anatomy, the catheter 11 is switched to the robotic insertion. This enables insertion of the catheter 11 in a more limited manner, which is performed at a slower, more leisurely speed to minimize causing abrasion and discomfort to the patient.

First, the manual insertion will be described in detail. Initially, as illustrated in FIG. 1, the posture and position of the support base 2 are adjusted such that the catheter 11 is directed at an insertion port of the subject (for example, the patient's oral cavity, nasal cavity, or the like). The posture and position of the support base 2 are adjusted by moving the arm 6. FIG. 1 illustrates the medical apparatus 1A in a state where the posture and position of the support base 2 have been adjusted. As illustrated in FIG. 2, the bending medical device 1 is attached to the tip of the guide rail 26 in the W direction.

Next, while the movement unlock button 205 on the attachment portion 201 of the base unit 200 is pressed and held down, the bending medical device 1 is moved in the Z direction relative to the guide rail 26. At this time, since the bending medical device 1 is locked so as not to be detached from the guide rail 26, the bending medical device 1 moves in the Z direction while still being attached to the guide rail 26. FIG. 3A illustrates the position of the bending medical device 1 after the manual insertion.

Next, the robotic insertion will be described. The cylinder 22 is protruded in the Z direction by the driving force of the motor 21 disposed in the support unit 20 of the support base 2. Accordingly, the moving stage 25 to which the bending medical device 1 is attached moves in the Z direction. FIG. 3B illustrates the position of the bending medical device 1 after the robotic insertion.

After inserting the catheter 11 into the inside of the patient as the subject, the operator performs operations such as observing, sampling, and excising on lung tissue. After completion of the operations, the operator removes the catheter 11 from the inside of the patient as the subject. The catheter 11 is removed by performing the procedure of insertion in reverse order, for example. More specifically, the support unit 20 of the support base 2 moves the moving stage 25 to which the bending medical device 1 is attached.

Subsequently, the operator manually moves the bending medical device 1 in the W direction while the bending medical device 1 is still attached to the guide rail 26, and removes the catheter 11 from the inside of the patient. During this manual removal, the bending medical device 1 can be moved in the W direction without pressing the movement unlock button 205 since the engaging teeth 27 and the movable engaging teeth 203 constitute a ratchet as described above.

As described above, according to the present exemplary embodiment, the medical apparatus 1A includes the moving stage 25 that supports the bending medical device 1 in a detachably attachable manner, and the support base 2 that includes the support unit 20 supporting the moving stage 25 to be linearly movable in predetermined directions (Z direction and W direction). Since the moving stage 25 supports the bending medical device 1 to be linearly movable in predetermined directions (Z direction and W direction), the operator can manually move the bending medical device 1 along the moving stage 25. Therefore, the operator does not need to perform operations such as attaching and detaching the bending medical device 1, and the operability of the bending medical device 1 can be improved.

According to the present exemplary embodiment, the moving stage 25 itself is moved by the driving force of the motor 21 of the support unit 20. Accordingly, a moving distance by the robotic insertion can be freely set regardless of the position of the bending medical device 1 relative to the moving stage 25 at the start of the manual insertion or a distance moved by the manual insertion. Therefore, it is possible to improve flexibility with regard to a structure of an insertion target and operability of the insertion.

According to the present exemplary embodiment, in the manual insertion or the manual removal, the bending medical device 1 moves in the Z direction or the W direction while being attached to the guide rail 26. At that time, on the bending medical device 1, the protrusions 202a of the pressing rollers 202 are disposed as lock members that lock the bending medical device 1 so as not to be detached from the moving stage 25, and the protrusions 202a engage with the protrusions 26a of the guide rail 26. Therefore, the bending medical device 1 does not unexpectedly come off the guide rail 26, and the bending medical device 1 can be safely moved in the Z direction and the W direction.

According to the present exemplary embodiment, the bending medical device 1 includes the movable engaging teeth 203 as a lock member that locks the bending medical device 1 so as not to linearly move relative to the moving stage 25, and the movable engaging teeth 203 engage with the engaging teeth 27. This prevents the bending medical device 1 from moving unexpectedly relative to the moving stage 25 at the time of the robotic insertion.

The above-described lock member that locks the bending medical device 1 so as not to linearly move locks only in the direction (Z direction) in which the bending medical device 1 is inserted into the subject, and does not lock the bending medical device 1 in the direction (W direction) in which the bending medical device 1 is removed from the subject. Therefore, when the operator wishes to quickly remove the bending medical device 1, the operator can remove the bending medical device 1 without pressing the movement unlock button. This improves operability of the manual removal.

In the present exemplary embodiment, the procedure by which the catheter 11 is inserted by the manual insertion and then by the robotic insertion has been described, but the present invention is not limited to this procedure. For example, the catheter 11 may be inserted first by performing the robotic insertion, then switching to the manual insertion, and again performing the robotic insertion. In addition, the removal of the catheter 11 is not limited to performing the procedure of insertion in reverse order, and may be performed by other procedures.

As described above, the bending medical device 1 is attachable to and detachable from the support base 2, and the connection between the bending medical device 1 and the control unit 3 is maintained even when the bending medical device 1 is detached from the support base 2 (detached state). In other words, the control unit 3 is always electrically connected to the bending medical device 1 regardless of an attachment/detachment state of the bending medical device 1 with regard to the support base 2 (moving stage 25). With this configuration, the operator can manually insert and remove the catheter 11 into and from the inside of the patient while holding the bending medical device 1 in the operator's hand. Therefore, it is possible to use the manual insertion and manual removal of the catheter 11 on the moving stage 25 in combination with the manual insertion and manual removal of the catheter 11 with the bending medical device 1 held by the operator in his or her hand. For example, the catheter 11 can be inserted by the manual insertion and the robotic insertion on the moving stage 25, and the catheter 11 can be manually removed while the bending medical device 1 is held by the operator in his or her hand. This further improves flexibility in inserting and removing the catheter 11.

In the present exemplary embodiment, the moving stage 25 is moved relative to the support unit 20 by the driving force of the motor 21 of the support unit 20, and the bending medical device 1 is manually moved relative to the moving stage 25. Alternatively, the moving stage 25 may be manually moved relative to the support unit 20, and the bending medical device 1 may be moved relative to the moving stage 25 by the driving force of a motor or the like. In the case of driving the bending medical device 1 by the motor 21 relative to the moving stage 25, the weight of a driven target is small, so a driving source can be downsized. On the other hand, operating forces for the manual insertion and manual removal are large, so the configuration can be changed according to the specifications to be prioritized.

In the present exemplary embodiment, the attachment/detachment unlock button 204 and the movement unlock button 205 are disposed. Alternatively, a single button may be disposed to serve as these buttons. For example, the button may be configured to release the movement lock when being half-pressed, and release the attachment/detachment lock when being fully pressed.

Although the present invention has been described above together with the exemplary embodiments, the present invention is not limited to the above-described exemplary embodiments, and modifications and the like are possible within the scope of the present invention.

The present invention is not limited to the above-described exemplary embodiments, and various modifications and variations can be made without departing from the spirit and scope of the present invention. Therefore, the following claims are appended to publicize the scope of the present invention.

According to the present invention, it is possible to improve the operability of a bending medical device.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. A medical apparatus comprising:
a bending medical device including a bendable body and a base part on which the bendable body is disposed; and
a support base including a moving stage configured to support the bending medical device in a detachably attachable manner and a support unit configured to support the moving stage so as to be movable linearly in a predetermined direction,
wherein the moving stage supports the bending medical device so as to be movable linearly in the predetermined direction.

2. The medical apparatus according to claim 1,
wherein the support unit includes a drive source, and
wherein the moving stage is moved relative to the support unit by a driving force of the drive source.

3. The medical apparatus according to claim 1, wherein the bending medical device is manually moved relative to the moving stage.

4. The medical apparatus according to claim 1, wherein the bending medical device includes a first lock member that locks the bending medical device so as not to be detached from the moving stage.

5. The medical apparatus according to claim 1,
wherein the moving stage has an elongated shape in the predetermined direction, and
wherein the bending medical device is attachable to the moving stage at any position in the predetermined direction.

6. The medical apparatus according to claim 1, wherein the bending medical device includes a second lock member that locks the bending medical device so as not to move relative to the moving stage.

7. The medical apparatus according to claim 6, wherein the second lock member locks the bending medical device so as not to move in a direction toward a subject and does not lock the bending medical device so as to move in a direction away from the subject.

8. The medical apparatus according to claim 6,
wherein the bending medical device includes a movement unlock member, and
wherein the second lock member locks the bending medical device so as not to move when an operator does not press the movement unlock member, and does not lock the bending medical device so as to move while the operator presses and holds down the movement unlock member.

9. The medical apparatus according to claim 1, further comprising a control unit electrically connected to the bending medical device and the support base,
wherein the control unit is always electrically connected to the bending medical device regardless of whether the bending medical device is attached to or detached from the moving stage.
